(19) United States
(12) Patent Application Publication
(10) Pub. No.: US 2014/0093966 A1
(43) Pub. Date: Apr. 3, 2014

(54) DEVICE FOR SUPPLY OF REACTANT LIQUIDS

(71) Applicants: Josef Find, Schwetzingen (DE); Alfred Haas, Eppelheim (DE); Armin Brenner, Eppelheim (DE); Denis Huertgen, Nussloch (DE)

(72) Inventors: Josef Find, Schwetzingen (DE); Alfred Haas, Eppelheim (DE); Armin Brenner, Eppelheim (DE); Denis Huertgen, Nussloch (DE)

(73) Assignee: HTE AG, the High Troughput Experimentation Company, Heidelding (DE)

(21) Appl. No.: 14/119,613

(22) PCT Filed: May 23, 2012
(86) PCT No.: PCT/EP2012/059553

§ 371 Date: Nov. 22, 2013

(30) Foreign Application Priority Data
May 24, 2011 (DE) 10 2011 102 361.9

(51) Int. Cl. B01L 3/00; G01N 33/24

(52) U.S. Cl. CPC B01L 3/56; G01N 33/241
USPC 436/37; 422/501

(57) ABSTRACT

The inventive device serves for simultaneous supply of nonvolatile reactant liquid to a plurality of mixing points or to a plurality of reactors, the device comprising a reservoir vessel, a supply line and a splitter which divides the supply line into a group of downstream lines. Each individual downstream line is functionally connected to one mixing point or one reactor and each is equipped with a restrictor element, the restrictor elements and at least parts of the downstream lines being in contact with a sheath having a temperature control unit.

The present invention relates to a device for supply of at least one reactant liquid, especially of a nonvolatile reactant liquid, to a plurality of mixing points or a plurality of reactors. The mixing points or the reactors form part of an arrangement which is used preferably in laboratory operation for high-throughput analysis of solid catalysts or for optimization of process conditions in high-throughput operation. High-throughput research serves to accelerate research and development processes, in order to reduce the duration of new development of a product or of a process before introduction to the market.

In this context, WO 2010/003661 A1 discloses, in general terms, the regulation of the fluid flows of individual capillaries or groups of capillaries in such apparatuses for high-throughput research.

One of the problems underlying the present invention is that of reducing the range of variation of mass balances in the catalytic conversion of reactant liquids, especially of nonvolatile reactant liquids, and of contributing to an improvement in the measurement data quality. A further problem is that of optimizing arrangements for high-throughput research such that they are better suited to prolonged operation.

The problem addressed by the invention and further problems are solved by providing a device for essentially simultaneous supply of at least one reactant liquid, especially of at least one nonvolatile reactant liquid, to a plurality of mixing points or to a plurality of reactors arranged in parallel in a catalysis apparatus. This device has at least: a reservoir vessel for at least one reactant liquid, especially for at least one nonvolatile reactant liquid; at least one feed line; at least one splitter (distributor) and a group of downstream lines (i.e. downstream of the distributor/splitter), wherein each downstream line in the group of downstream lines is functionally connected to one restrictor element, and the entirety of the restrictor elements and at least parts of the downstream lines is/are in contact, preferably in direct physical contact, with a body having a density of >1 g/cm³ and a heat capacity of >100 J/kg·K.

Preferably, this body having a density of >1 g/cm³ and a heat capacity of >100 J/kg·K has a metal core in which there is elevated conduction of heat. Preference is given here to aluminum or steel. In this context, the storage capacity of said body for heat has a favorable effect on the efficacy of the restrictors. The body or the metal core is preferably surrounded by a heat-insulating layer. The restrictor elements are preferably within a gap between the metal core and insulating sheath.

In a preferred embodiment, the restrictor elements are capillary restrictors. The capillary restrictors and parts of the downstream lines which are in (direct physical) contact with the body having a density of >1 g/cm³ and having a heat capacity of >100 J/kg·K are preferably heated with the temperature control unit to a temperature within a range from 30° C. to 200° C. Preferably, the temperature is within a range from 50° C. to 180° C. and further preferably within a range from 60° C. to 160° C.

Preferably, in the context of the present invention, the body having a density of >1 g/cm³ and a heat capacity of >100 J/kg·K has a high constancy of temperature, with deviations in the temperature preferably not greater than ±1 K per meter of length of restrictor, preferably capillary restrictor. Further preferably, the deviation is not greater than ±0.5 K per meter of length. The temperatures of adjacent restrictors should preferably not differ by more than 0.5 K, in order to achieve maximum equality of distribution of fluid streams. As has been found, such constancy of temperature is particularly advantageous especially for nonvolatile reactant liquids. Further preferably, the temperature difference should be equal to or less than 0.3 K. Even further preferably, the temperature difference should be equal to or less than 0.1 K. It has been found that, surprisingly, the inventive body having the comparatively high density and heat capacity has a great influence on the improvement of process control and the associated measurement data quality. This is especially true compared to an arrangement in which the temperature of the restrictors is controlled only or primarily via air circulation.

In a preferred embodiment, the inventive device is incorporated into an apparatus for high-throughput research, preferably for catalyst testing, with each individual downstream line connected either to one mixing point or to one reactor inlet. Each individual mixing point preferably has a fluid supply for gaseous components. The mixing point serves to mix or to combine a reactant liquid, especially a nonvolatile reactant liquid, with one or more gaseous components.

It is preferably a characteristic feature of nonvolatile liquids in the context of the present invention that at least 50% by weight, preferably more than 70% by weight and further preferably more than 90% by weight of liquid has a boiling point greater than 350° C. at standard pressure.

The fluid combined in the individual mixing points is preferably passed in each case to a reactor. Alternatively, the reactant liquid, preferably nonvolatile reactant liquid, can also be conveyed proceeding from the respective line downstream of the splitter/distributor directly into a reactor. If reactant liquid, preferably nonvolatile reactant liquid, is conveyed directly to the individual reactors by means of the individual downstream lines, it is possible that the reactant liquid, preferably the nonvolatile reactant liquid, is mixed with gaseous fluid at the reactor inlet or in the region of the reactor inlet.

The present invention also relates to a process for essentially simultaneous supply of at least one reactant liquid, especially a nonvolatile reactant liquid, to a plurality of mixing points or to a plurality of reactors, using an inventive device.

In a preferred embodiment, at least part of the inventive device for the supply of at least one reactant liquid, preferably at least one nonvolatile reactant liquid, is disposed in an air circulation oven or in an oven chamber.

With regard to the dimensions of the oven chambers, it is preferable that the dimensions of the oven chambers are configured according to factors including how many downstream lines are in (physical) contact with an inventive body having a density of >1 g/cm³ and a (specific) heat capacity of >100 J/kg·K, and what dimensions the individual restrictor elements have.

Such an inventive body preferably is in contact, preferably in direct physical contact, with at least four or more downstream lines having restrictor elements, preferably with six or more downstream lines having restrictor elements, further preferably with between ten and one hundred downstream lines having restrictor elements.

Such an inventive body in contact with twenty downstream lines having restrictor elements can preferably be disposed in an oven chamber, the internal volume of which is in the range from 0.5 to 150 I. Preferably, the internal volume of one oven chamber is in the range from 0.7 to 50 I; further preferably, the internal volume of one oven chamber is in the range from 0.9 to 10 I.

With regard to the capillary restrictors disposed in the downstream lines, it is preferable that these have steel as a material, preferably as the predominant material, and further preferably consist essentially of steel. The length of the capillary restrictors is preferably within a range from 0.2 m to 6 m, more preferably within a range from 0.5 m to 3 m. The internal diameter of the individual capillary restrictors is preferably within a range from 50 to 750 μm, preference being given to an internal diameter within a range from 100 μm to 500 μm. The ratio of the cross-sectional area of the downstream line (Q_{FU}) to the cross-sectional area of the capillary restrictors (Q_{KR}), i.e. Q_{FU}/Q_{KR}, is preferably ≧3, and further preferably Q_{FU}/Q_{KR}≧5.

Especially if the capillary restrictors have a length of more than 0.3 m, these capillary restrictors are wound around a core of the inventive body or fitted into a spiral mold. In this case, the core and/or the spiral mold is a body having heat capacity in the context of the present invention.

The inventive device for supply of at least one reactant liquid, especially of a nonvolatile reactant liquid, is preferably operated in conjunction with a catalysis apparatus in order to introduce said reactant liquid essentially simultaneously over a long period with high accuracy and higher reproducibility in reactors connected in parallel in a catalysis apparatus. The product streams generated in the reactors are subjected to one or several analyses in order to determine the efficacy of the catalysts and/or the optimal process conditions as a function of the objective of the analysis.

The preferred field of use of the inventive device relates to catalytic studies which are conducted at a liquid hourly space velocity (LHSV) in the range from 0.05 to 10 h⁻¹, further preference being given to an LHSV of 0.2 to 3 h⁻¹. Accordingly, the device is preferably used in conjunction with reactors having an internal volume in the range from 0.2 ml to 100 ml. The rectors preferably have an internal volume of 0.5 ml to 50 ml.

In a preferred embodiment, the reservoir vessel for the at least one reactant liquid, especially nonvolatile reactant liquid, is equipped with a stirrer element and has a separate heating device. The reactant liquid, especially the nonvolatile reactant liquid, is transferred from the reservoir vessel to the splitter and through the restrictor elements preferably by means of pressurization, and further preferably using a pump. The pump may be selected from the group of metering pumps, HPLC pumps. It is possible to meter the reactant liquid, especially nonvolatile reactant liquid, into reactors whose internal reactor pressure is in the range from 1 to 250 bar, the internal reactor pressure further preferably being within a range from 2 to 180 bar.

The term “reactant liquid” in the context of the present invention refers to substances which are present in the form of liquids and can enter into a chemical reaction. The reactant liquids are preferably nonvolatile reactant liquids. More particularly, the nonvolatile reactant liquids are selected from the group of oils, heavy oils, waxes, VGO (vacuum gas oil) and mixtures thereof. They are preferably hydrocarbonaceous compounds which may also comprise nitrogen- and sulfur-containing components. In the context of the present invention, it is possible that the nonvolatile reactant liquids are present as solids at room temperature. It is preferably a characteristic feature of nonvolatile liquids in the context of the present invention that at least 50% by weight, preferably more than 70% by weight and further preferably more than 90% by weight of the liquid has a boiling point greater than 350° C. (in each case at standard pressure).

If the nonvolatile reactant liquids to be examined comprise solid particles in the form of deposits or coke, these deposits are preferably removed by a filtration step. The capillary elements of a microscale metering device, because of the small dimensions, can be blocked by solid particles, which leads to impairment of function. Solid particles having a size in the region of about 1 μm generally cannot be removed by the filtration operation. In this respect, it is not advisable for such reactant liquids (comprising particles) to select too small a capillary diameter. At the same time, it is advantageous to select the capillaries with maximum length and to contact them with the inventive body.

The diameter of the restriction capillaries is thus, in a preferred embodiment, determined by the size of solid particles, in which case the diameter of the capillaries should preferably be at least ten times greater than the diameter of the smallest non-removable solid particles, i.e. at least ten times greater than 1 μm, i.e. greater than 10 μm.

The term “gaseous fluid” comprises fluids which are in the gaseous state under reaction conditions. These may either be reactant components which take part in the reaction or inert gas components which serve as a carrier gas or calibration gas standard.

The term “high-throughput research” in the context of the present invention refers particularly to catalyst test benches having a plurality or a multitude of reactors arranged in parallel in the dimensions of what are called bench-scale plants. This area of plant construction differs from the area of microscale reactor technology in that, in the system construction of present relevance, preferably no components having dimensions below 1 mm are used.

Microscale reactor technology is based on the use of components having very small dimensions. The lines and channels have dimensions in the sub-millimeter range. The sample amounts used of solid catalysts to be examined are within a range below 100 mg. The more complex the chemical reactions to be evaluated by means of the catalytic experiments, the more critical is the use of microscale reactor technology. In many cases, it is impossible to obtain meaningful and robust data.

The present invention also relates to the combination of components from the field of microscale reaction technology—in the form of the inventive device—with pilot plants or bench-scale plants, which are equipped with individual, mutually independent reactors. The success of this combination is apparent from the data quality, which is expressed by the mass balances or material recovery rate, and which has been crucially improved by means of the present device.

On the basis of the present invention, it is possible to distinctly improve the data quality of catalysis data which are obtained by means of bench-scale plants or laboratory pilot plants. As a result of the higher data quality, the number of costly catalytic studies on a larger scale in large pilot plants can be greatly reduced. Overall, it is possible to accelerate research operations, or to greatly restrict energy consumption in experiments on the large scale.

More particularly, in the area of nonvolatile reactant liquids, the inventive device is of great significance.

With reference to FIG. 3, it is apparent that the viscosity of n-dodecane, a nonvolatile reactant liquid, is highly dependent on the temperature. n-Dodecane has high structural viscosity within the temperature range from 260 K to 400 K. Because of the high temperature dependence of the viscosity of reactant liquids and especially of nonvolatile reactant liquids, the thermal coupling and monitoring of the restriction elements of the microscale metering device is of crucial significance, in order to achieve exact homogeneous distribution of the reactant liquid, preferably nonvolatile reactant liquid.

The present invention also relates to a combination of an inventive device for parallel metered addition of liquids with a catalysis apparatus having reactors arranged in parallel, the reactors preferably being of the size of conventional laboratory reactors, or else taking the form of reactors in a small pilot plant.

FIG. 3 shows the viscosity values for a permanent gas (methane) and a liquid (n-dodecane) as a function of temperature.

This shows that the viscosity of methane within the range from 300 to 400 K rises from about 11 to 15 μPas. Within the same temperature range, the viscosity of the liquid falls from 1500 to 500 μPas, meaning that the viscosity of the liquid decreases by about a factor of 3. The temperature-dependent profile also shows that the decrease in viscosity in the range between 270 and 300 K is within the same order of magnitude as in the range between 300 and 400 K. This greatly temperature-dependent range of viscosity is referred to as the “structurally viscous range”. Within this highly temperature-dependent range, homogeneous temperature control is of even greater significance than in the less significantly temperature-dependent viscosity ranges. In these highly temperature-dependent ranges, the inventive unit can be used particularly advantageously.

FIGS. 4 and 5 show embodiments of capillary holders which form part of an inventive device. The term “passive heating” in the context of the present invention means that the device can be installed in an air circulation oven and is heated simultaneously by the circulating air in the oven. The capillaries are either in a form wound around a core (FIG. 4) or have been introduced into individual capillary compartments (FIG. 5). The capillary compartments are preferably between two adjacent lands (6, 7).

In the embodiment of FIG. 4, heat conductors in the form of half-shells (3, 3′) are present in the outer region of the holder. Between the metal core (1) and insulation half-shells are two half-shells of insulation material (2, 2′). The heat-conducting housing shells (3, 3′), the core (1) and (5) are functionally connected to heat-conducting plates (4, 4′) at the ends. In contrast, in FIG. 5, the heat-conducting half-shells are disclosed between core (1) and insulator half-shells (2, 2′).

In an alternative embodiment, the housing half-shells are replaced by a tube slotted on one side. Otherwise, preferably a gap in the range from 1 to 3 mm is present between the half-shells. This gap serves for passage of the ends of the capillary lines.

In a preferred embodiment, the temperature of the capillary device shown in FIG. 5 can be controlled very homogeneously by a single heating cartridge. The heating cartridge is preferably centered. It is preferable that the embodiment shown in FIG. 5 is either installed in an air circulation oven or is operated outside an oven. If the housing is operated in an air circulation oven, the temperature of the capillary holder is preferably higher than the temperature of the air circulation oven, in which case the temperature difference from the air circulation oven is preferably greater than 20 K, further preferably greater than 10 K and still further preferably greater than 5 K.

### BRIEF DESCRIPTION OF THE FIGURES

FIG. 1 shows a graph of the mass balances (the figure on the ordinate relates to % by weight of heavy oil) which were determined in the separators on the side of the product-collecting system after the simultaneous metered addition of heavy oil into sixteen reactors connected in parallel. Comparative examples CE1 to CE3 reflect the values which were obtained in the case of metered addition according to the prior art (temperature control only via the air circulation). The inventive example IE1 shows the values which have been obtained by means of the inventive device (temperature control via the inventive body).

FIG. 2 shows the graph of the mass balances as obtained in an inventive device as a function of time over a period of fifteen days. The feed was metered into sixteen reactors arranged in parallel by means of the inventive device (at a temperature of 90° C.). The amounts of feed accommodated in the downstream reactors were determined gravimetrically. Each individual measurement point represents a value which has been determined by average formation over the sixteen mass balances. The vertical bars indicate the standard deviation which has been obtained in the average formation over the sixteen individual values of the respective measurement days.

FIG. 3 shows the graph of the viscosities of methanol and n-dodecane as a function of temperature for a temperature range from 260 K to 400 K. The viscosity values of methane are represented as triangles and the viscosity values of n-dodecane as plus symbols. The viscosity values are reported in the unit [μPa*s], the values on the left-hand ordinate relating to n-dodecane and the numerical values on the right-hand ordinate to methane.

FIG. 4 shows the schematic diagram of a cylindrical version of a multilayer capillary holder with a metal core (1), which is suitable for the passive heating.

FIG. 5 shows a similar embodiment to the diagram in FIG. 4, except that the metal core (4) has been replaced by a metal cylinder with recessed chambers (6, 7).

### LIST OF REFERENCE NUMERALS

1—core
2, 2′—thermal insulation or half-shells
3, 3′—heat conductors
4, 4′—end plates, heat conductors
5—core with grooves
6, 7—adjacent lands

### WORKING EXAMPLES

The examples adduced relate to the supply and conversion of nonvolatile reactant liquids in a high-throughput apparatus with sixteen reactors arranged in parallel, and serve to illustrate the invention. The reactions selected here were hydrocracking reactions.

In accordance with the illustrative embodiment, the nonvolatile reactant liquid used was a crude feed which was obtained as a residue in an atmospheric distillation. The melting point of the crude feed was 86° C. and the boiling point was 370° C. The crude feed was converted in the presence of hydrogen in a trickle bed process, using nitrogen as the carrier gas. The sixteen reactors were each charged with 10 ml of solid catalyst. The reactant liquid was supplied to the individual reactors with an LHSV of 1.5 h⁻¹.

The amount of liquid product which had been accommodated in the separators downstream of the reactors over a given period was recorded gravimetrically. The product composition was determined by means of gas chromatography.

An experimental setup in which the inlet for liquid reactant was divided by means of a splitter into downstream lines provided with restrictor elements was used, using a setup analogous in principle to that from the PCT application WO 2005/063372. However, the inventive device was used in addition.

In the comparative example, the restrictor elements and parts of the downstream lines were accommodated directly in an air circulation oven chamber without the inventive body. Nonvolatile reactant liquid was introduced simultaneously into sixteen reactors and the product stream obtained in the individual reactors was characterized analytically in order to determine the mass balance, with variation of the temperature of the air circulation oven chambers. The temperatures selected here for the air circulation oven chambers for heating of the restrictor elements were 88° C., 90° C. and 92° C. The start temperature was 25° C.

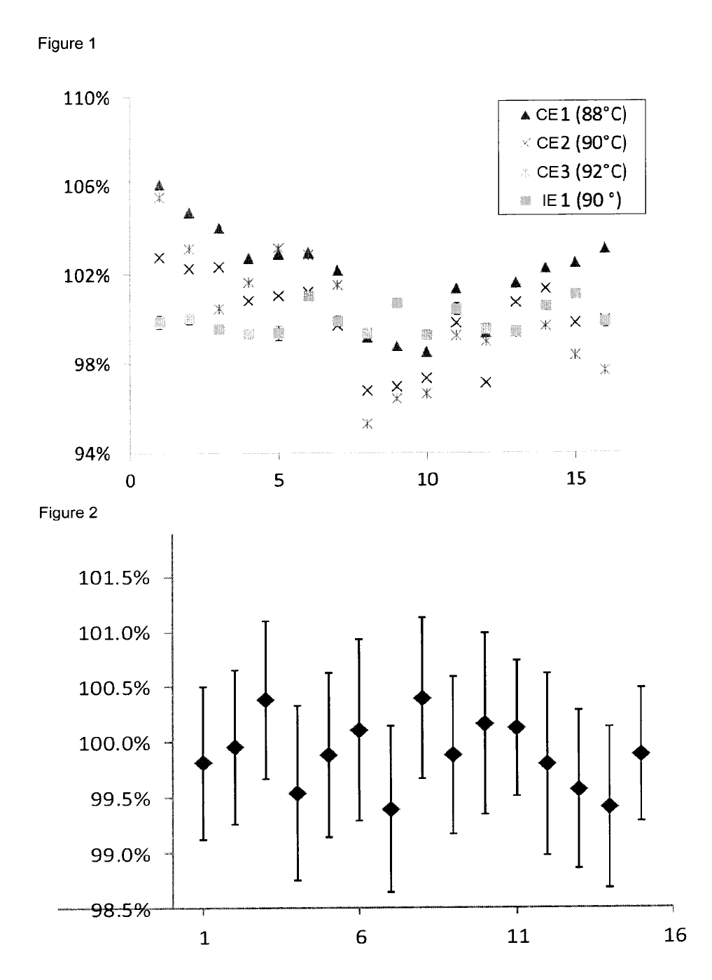

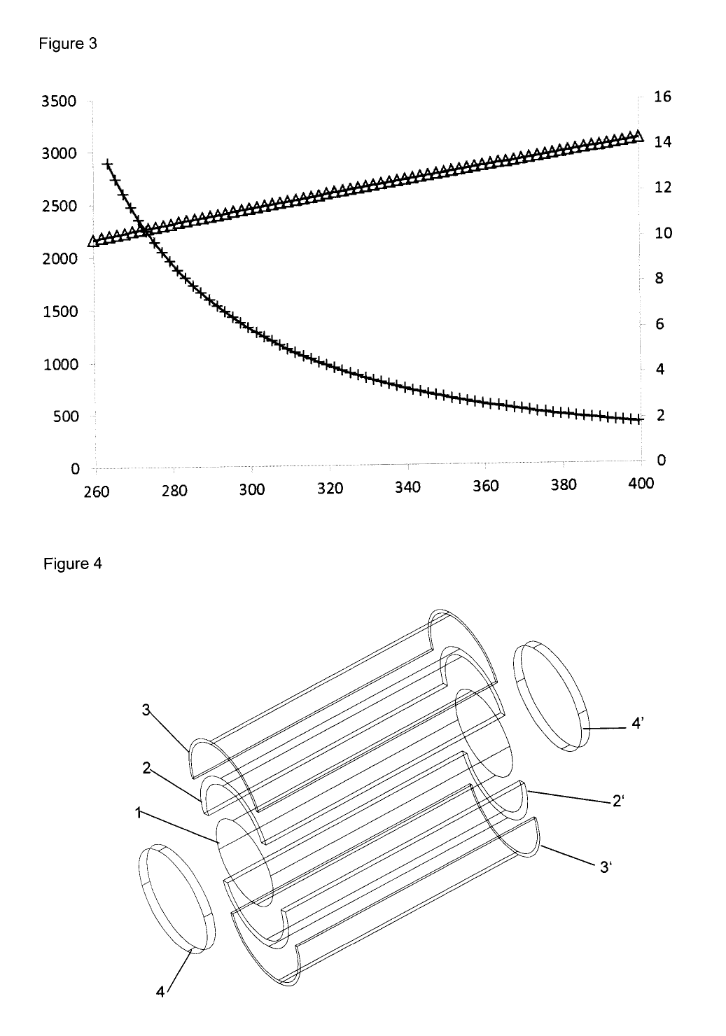

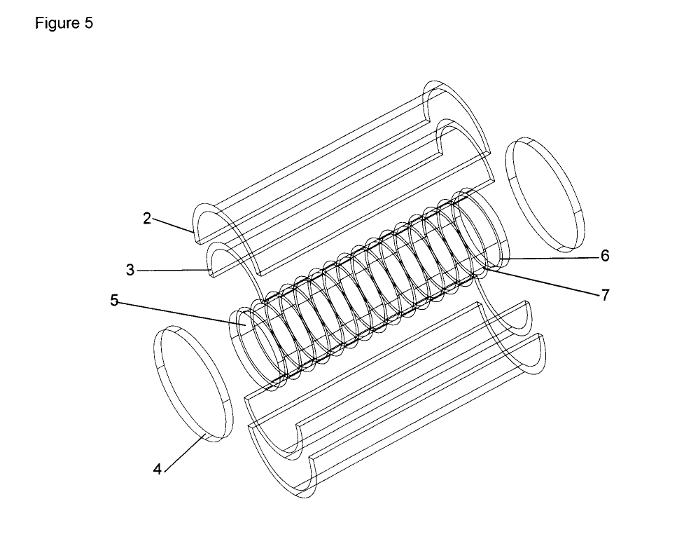

The mass balances which were determined after the supply of reactant liquid at different temperatures of the air circulation chambers are shown in FIG. 1.

### Inventive Example 1

In inventive example 1, the studies of reactant liquid supply were conducted in an inventive device, which was otherwise accommodated in the same air circulation oven chamber as in the comparative example. The restrictor elements consisted of stainless steel capillaries having a length of 1.5 m and had an internal diameter of 150 μm. The restrictor elements were wound around a metal core and sheathed by silicone heating mats. Three thermocouples for temperature monitoring were provided in the sheath. The temperature of the sheathed restrictor elements was regulated with a digital regulator.

The results show that the range of variation in the mass balance is distinctly reduced using the inventive device compared to the prior art. According to the prior art, the range of variation in the mass balances is approximately within the range of ±3%. Using the inventive device, the range of variation of the mass balances, in contrast, is within a range less than or equal to ±1.5%.

In addition, long-term studies were conducted, in which the reactant liquid was conveyed into the reactors of the catalysis apparatus over a period of seven weeks. The mass balances determined here show that a very low range of variation is present by means of the inventive device.

The result of the studies is shown in FIGS. 1 and 2. What are shown there are those amounts of nonvolatile reactant liquid which have been accommodated in respective product-collecting vessels after the metered addition of reactant liquid into sixteen reactors. The outlet line from each reactor is connected to one product-collecting vessel. The figure for the amount of material recovered is given in percent.

1. A device for essentially simultaneous supply of at least one reactant liquid, to a plurality of mixing points or to a plurality of reactors, the device comprising:
at least one reservoir vessel suitable for reactant liquids;
at least one supply line;
at least one splitter; and
a group of downstream lines,
wherein:
each downstream line in the group of downstream lines is functionally connected to one restrictor element; and
the entirety of restrictor elements and at least parts of the downstream lines are in direct physical contact with a body having a density of >1 g/cm³ and a heat capacity of >100 J/kg·K.

2. The device according to claim 1, wherein the restrictor elements are capillary restrictors and are heated together with the body to a temperature within the range from 30° C. to 200° C.

3. The device according to claim 1, wherein the device is part of a catalysis apparatus for testing solid catalysts.

4. The device according to claim 1, wherein some or all of the device is disposed in an air circulation oven or in an oven zone of a multichamber oven.

5. The device according to claim 2, wherein:
the capillary restrictors comprise stainless steel;
the length of each capillary restrictor is within a range from 0.2 to 6 m;
the internal diameter of each capillary restrictor is within a range from 50 to 750 μm; and
a ratio of the cross-sectional area of the downstream lines to the cross-sectional area of the capillary restrictors Q_{FU}/Q_{KR} is ≧3.

6. The device according to claim 1, wherein:
at least one reactant liquid supplied has an LHSV in the range from 0.05 to 10 h⁻¹; and
each individual reactor functionally connected to the device has a volume in the range from 0.2 to 100 ml.

7. The device according to claim 1, wherein:
the reservoir vessel is equipped with a stirrer element and has a separate heating device; and
a feed line from the reservoir vessel to the splitter is functionally connected to a pump.

8. A process comprising essentially simultaneously supplying, with the device of claim 1, at least one reactant liquid to a plurality of mixing points or to a plurality of reactors.

9. (canceled)

10. The device of claim 1, wherein the device is configured to essentially simultaneously supply at least one reactant liquid to a plurality of mixing points or to a plurality of reactors.

11. The device of claim 10, wherein the reactant liquid is a nonvolatile reactant liquid.

12. The device of claim 10, wherein the device is configure to essentially simultaneously supply the at least one reactant liquid to a plurality of reactors arranged in parallel within a catalysis apparatus.

13. The device according to claim 1, wherein the restrictor elements are capillary restrictors and are heated together with the body to a temperature within the range from 60° C. to 160° C.

14. The device according to claim 2, wherein the device is part of a catalysis apparatus for testing solid catalysts.

15. The device according to claim 5, wherein the internal diameter of each capillary restrictor is within a range from 100 μm to 500 μm.

16. The device according to claim 5, wherein the ratio of the cross-sectional area of the downstream lines to the cross-sectional area of the capillary restrictors Q_{FU}/Q_{KR} is ≧5.

17. The device according to claim 6, wherein at least one reactant liquid supplied has an LHSV in the range from 0.2 to 3 h⁻¹.

18. The device according to claim 6, wherein each individual reactor functionally connected to the device has a volume in the range from 0.5 to 50 ml.

19. The device according to claim 5, wherein the length of each capillary restrictor is within a range from 0.5 to 3 m.
